(19) United States
(12) Patent Application Publication
(10) Pub. No.: US 2005/0171462 A1
(43) Pub. Date: Aug. 4, 2005

(54) Identification means for swab applicator

(76) Inventor: Garry Tsaur, Rowland Heights, CA (US)

Correspondence Address: JOE NIEH, 18760 E. AMAR ROAD #204, WALNUT, CA, 91789, US

(21) Appl. No.: 10/769,267

(22) Filed: Jan. 30, 2004

(51) Int. Cl. A61M 35/00

(52) U.S. Cl. 604/1

(57) ABSTRACT

An identification means for swab applicator that enables a person to distinguish one end of the swab applicator from the other end even with impaired vision or in low light conditions. In addition, the identification means enables one to accurately determine the position of a fracture line on the swab applicator for release of the fluid enclosed within the swab applicator. The identification means for swab applicator comprises of a raised ring or a ring of channel on the applicator tip positioned near the fracture line on the swab applicator that can be seen and felt.

### BACKGROUND

1. Field of Invention

The present invention relates generally to identification means for swab applicator. More specifically, the present invention relates to identification means positioned on the swab or tip.

2. Description of Related Art

Swab applicator generally comprises of an elongated tubular body with an applicator tip affixed to one or both of its ends. The elongated tubular body may be made of wood, paper, or plastic and may be a solid member or a hollow tube. The applicator tip may be a foam tip, a sponge, a wad of cotton, or a brush. Swab applicator with a hollow tubular body may also enclose a fluid with one or both ends sealed.

Swab applicator that contains a fluid may have one or both ends sealed with an applicator tip affixed to one or both ends. An opening means such as a fracture line may be provided near one end such that when the hollow tubular body is broken open at the fracture line, the fluid will flow out of the hollow tubular body for application. When both ends of the hollow tubular body are each wrapped by an applicator tip, it becomes virtually impossible to distinguish which end of the container has the fracture line that needs to be broken open to release the fluid. In addition, even when only one end is wrapped by the applicator tip, the position of the fracture line cannot be seen or accurately determined. It is a hit-or-miss when bending the hollow tubular body in an attempt to locate the fracture line. Furthermore, if the hollow tubular body with both ends wrapped by an applicator tip is being used by a person with impaired vision or in low light condition, it also becomes virtually impossible to distinguish one end of the hollow tubular body from the other even if one end is marked with a different color applicator tip. The position of the fracture line also cannot be determined in these situations.

### BRIEF SUMMARY OF THE INVENTION

The present invention is an identification means for swab applicator that enables a person to distinguish one end of the swab applicator from the other end even with impaired vision or in low light conditions. Furthermore, the identification means enables one to accurately determine the position of a fracture line on the swab applicator for release of the fluid enclosed within the swab applicator. In the preferred embodiment, the identification means for swab applicator comprises of a raised ring embossed on the applicator tip positioned near the fracture line on the swab applicator that can be seen and felt. In another embodiment, the identification means for swab applicator comprises of a ring of channel around the applicator tip positioned near the fracture line on the swab applicator that can be seen and felt.

### BRIEF DESCRIPTION OF THE DRAWINGS

FIG. 1 shows the preferred embodiment of the identification means for swab applicator.

FIG. 2 shows another embodiment of the identification means for swab applicator.

### DETAILED DESCRIPTION OF THE PREFERRED EMBODIMENT

The following description and figures are meant to be illustrative only and not limiting. Other embodiments of this invention will be apparent to those of ordinary skill in the art in view of this description.

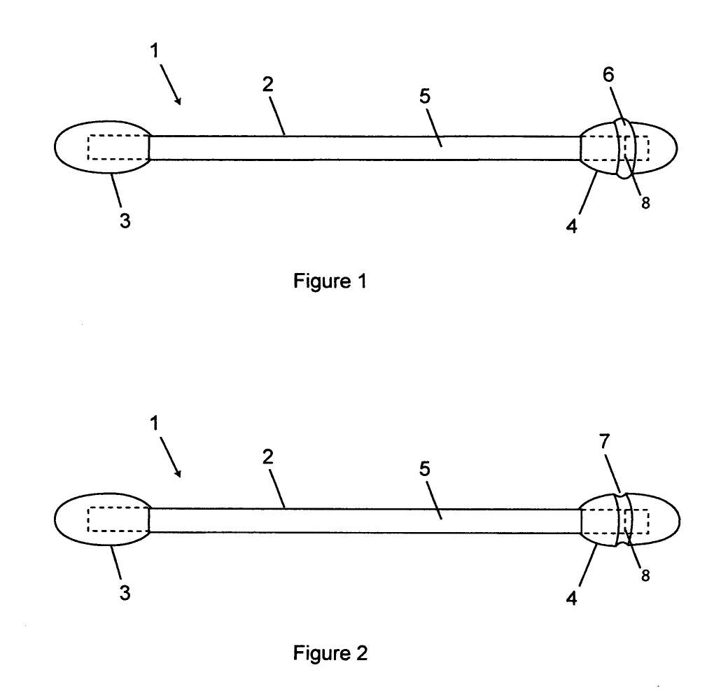

FIG. 1 shows a preferred embodiment of identification means for swab applicator. The swab applicator 1 comprises a hollow elongated tubular body 2 with an applicator tip 3, 4 affixed to each of its two ends and encloses a fluid 5 within its hollow elongated tubular body 2. A fracture line 8 is formed near one end of the hollow elongated tubular body 2 to allow the elongated tubular body 2 to be broken open at the fracture line 8. In the preferred embodiment, the identification means is a raised ring 6 embossed on the applicator tip 4 that can be seen and felt.

The raised ring 6 is position near the fracture line 8 on the elongated tubular body 2. The raised ring 6 around the applicator tip 4 can be painted with a color that can be easily seen by someone with normal or corrected vision under normal lighting conditions. The raised ring 6 can also be felt by anyone without looking at the swab applicator 1. One can easily distinguish the end of the swab applicator 1 with the fracture line 8 from the other end by simply touching the applicator tips 3, 4 to locate the applicator tip 4 with the raised ring 6. Therefore, a person with impaired vision or a person using the swab applicator 1 in low light conditions will be able to positively distinguish the end of the swab applicator 1 with the fracture line 8 from the other end and use the swab applicator 1.

In another embodiment of the identification means for swab applicator, the identification means comprises of a ring of channel 7 around the applicator tip 4 in the form of a depression that can be seen and felt. The ring of channel 7 is position near the fracture line 8 on the elongated tubular body 2. The ring of channel 7 around the applicator tip 4 can be painted with a color that can be easily seen by someone with normal or corrected vision under normal lighting conditions. The ring of channel 7 can also be felt by anyone without looking at the swab applicator 1. One can easily distinguish the end of the swab applicator 1 with the fracture line 8 from the other end by simply touching the applicator tips 3, 4 to locate the applicator tip 4 with the ring of channel 7. Therefore, a person with impaired vision or a person using the swab applicator 1 in low light conditions will be able to positively distinguish the end of the swab applicator 1 with the fracture line 8 from the other end and use the swab applicator 1.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An identification means for swab applicator comprising:
an elongated tubular housing with two ends and a fracture line formed near one end;
an applicator tip affixed to said end of said elongated tubular housing with said fracture line;
a fluid disposed within said elongated tubular housing; and
a raised ring formed on said applicator tip disposed near said fracture line;
wherein said raised ring can be seen and felt by a person.

2. An identification means for swab applicator as in claim 1, wherein said raised ring is a different color than said applicator tip.

3. An identification means for swab applicator comprising:
an elongated tubular housing with two ends and a fracture line formed near one end;
an applicator tip affixed to said end of said elongated tubular housing with said fracture line;
a fluid disposed within said elongated tubular housing; and
a ring of channel formed on said applicator tip disposed near said fracture line;
wherein said raised ring can be seen and felt by a person.

4. An identification means for swab applicator as in claim 3, wherein said ring of channel is a different color than said applicator tip.

5. An identification means for a swab applicator with an elongated tubular housing with two ends and a fracture line formed near one end and with an applicator tip affixed to said end with said fracture line with a fluid disposed within said elongated tubular housing wherein the improvement comprises of an identification means positioned on said applicator tip near said fracture line.

6. An identification means for swab applicator as in claim 5, wherein said identification means comprises of a raised ring around said applicator tip near said fracture line.

7. An identification means for swab applicator as in claim 6, wherein said raised ring has a different color than said applicator tip.

8. An identification means for swab applicator as in claim 5, wherein said identification means comprises of a ring of channel around said applicator tip near said fracture line.

9. An identification means for swab applicator as in claim 8, wherein said ring of channel has a different color than said applicator tip.
